(19) United States
(12) Patent Application Publication
(10) Pub. No.: US 2007/0027214 A1
(43) Pub. Date: Feb. 1, 2007

(54) ORALLY ADMINISTERED AGENT FOR IMPROVING SKIN CONDITION

(75) Inventors: Miho Komatsu, Tsukuba-shi (JP); Koji Morishita, Tsukuba-shi (JP); Akemi Ogawa, Ichikawa-shi (JP); Goro Hori, Tsuchiura-shi (JP)

Correspondence Address: DARBY & DARBY P.C., P. O. BOX 5257, NEW YORK, NY, 10150-5257, US

(73) Assignee: Kyowa Hakko Kogyo Co., Ltd., Tokyo (JP)

(21) Appl. No.: 11/421,875

(22) Filed: Jun. 2, 2006

(30) Foreign Application Priority Data
Jul. 28, 2005 (JP) 2005-218759

(51) Int. Cl. A61K 31/198

(52) U.S. Cl. 514/564

(57) ABSTRACT

It is desirable to provide a medicament and a nutritional food to improve skin conditions and to create fulfilling life for people who are not satisfactory about their skin conditions. Specifically, one object of the present invention is to provide an orally administered agent, a food and drink, or a food additive which can be used for improving skin condition. An orally administered agent, a food and drink, or a food additive for improving skin condition, comprising ornithine or a salt thereof as an active ingredient can be provided according to the present invention.

### BACKGROUND OF THE INVENTION

### Incorporation by Reference

The present application claims priority under 35 U.S.C. §119 to Japanese Patent Application No. 218759/2005 filed on Jul. 28, 2005. The content of the application is incorporated herein by reference in its entirety.

1. Field of the Invention

The present invention is related to an orally administered agent; a food and drink; and a food additive for improving skin condition, which comprises ornithine or a salt thereof as an active ingredient.

2. Description of the Related Art

Many people are not satisfactory about their skin conditions because skin is always exposed to a variety of stimuli from inside body and outside environment.

In general, cosmetic products containing of components, which may improve skin conditions, are applied to improve skin conditions. In addition, a bath agent, a drug, food and the like are applied to improve skin conditions.

Ornithine is being used, mostly in the U.S., as a food additive to strengthen muscle formation by letting the body secrete growth hormone or to prevent obesity by enhancing basal metabolism. Further, ornithine is used to produce L-ornithine-L-asparate which is a medicament used to improve a liver disorder in Europe.

Known compositions composed of ornithine having activities on skin are a cosmetic composition which enhances hair growth (Japanese Published Unexamined Patent Application No. 502509/1996) and a topical anti-skin cancer pharmaceutical preparation (Japanese Published Unexamined Patent Application No. 512410/1999).

### SUMMARY OF THE INVENTION

It is desirable to provide a medicament and a nutritional food to improve skin conditions and to create fulfilling life for people who are not satisfactory about their skin conditions. Specifically, one object of the present invention is to provide an orally administered agent, a food and drink, or a food additive which can be used for improving skin condition.

The present invention relates to the following aspects (1) to (6):
(1) an orally administered agent for improving skin condition which comprises ornithine or a salt thereof as an active ingredient.
(2) the agent according to (1), wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.
(3) a food and drink, or a food additive for improving skin condition which comprises ornithine or a salt thereof as an active ingredient.
(4) the food and drink, or the food additive according to (3), wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.
(5) a method for improving skin condition which comprises administering or ingesting ornithine or a salt thereof to a subject in need thereof.
(6) the method according to (5), wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.

### BRIEF DESCRIPTION OF THE DRAWINGS

FIG. 1 is scale graphs expressing questionnaires for evaluation using Visual Analogue Scale (VAS) method. Each end of the segment has a criterion of expression.

FIG. 2 is a graph showing improvements in skin condition by the ingestion of ornithine. The vertical axis shows an average improvement ratio (%) of each criterion of skin conditions.

### DETAIL DESCRIPTION OF THE INVENTION

Ornithine as applied in the present invention includes L-ornithine and D-ornithine, preferably L-ornithine.

Ornithine can be obtained by a chemical synthetic method or a fermentation method. Also, ornithine is commercially available.

A chemical synthetic method can be found in, for example, Coll. Czechoslov. Chem. Commun., 24, 1993 (1959).

A fermentation method is disclosed, for example, in Japanese Published Unexamined Patent Application Nos. 24096/78 and 119194/86.

L-ornithine and D-ornithine can be also purchased from, for example, Sigma Aldrich Company.

Salts of ornithine include acid addition salts, metal salts, ammonium salts, organic amine addition salts, amino acid addition salts and the like.

The acid addition salts include inorganic acid salts such as hydrochloride, hydrosulfate, nitrate and phosphate; and organic acid salts such as acetate, maleate, fumarate, citrate, malate, lactate, α-ketoglutarate, gluconate and caprylate.

The metal salts include alkali metal salts such as sodium salt and potassium salt; alkaline earth metal salts such as magnesium salt and calcium salt; aluminum salt, zinc salt and the like.

Ammonium salts include salts of ammonium, tetramethylammonium and the like.

Organic amine addition salts include salts of morpholine, piperidine and the like.

Amino acid addition salts include salts of glycine, phenylalanine, lysine, aspartate, glutamate and the like.

Among the above salts of ornithine, hydrochloride, citrate, malate, α-ketoglutarate and aspartate are preferably applied, but one of the remaining salts or two or more of the above salts can be arbitrarily used.

In addition to ornithine and its salt, a proper additive for each application can be added to an orally administered agent, a food and drink, or a food additive of the present invention.

The above additive includes amino acids such as valine, leucine, isoleucine, arginine, lysine, glutamine, alanine, serine, glycine, cistein and threonine, and the like.

Skin conditions can be improved by administration or ingestion of an orally administered agent, a food and drink, or a food additive according to the present invention.

Improvements in skin condition according to the present invention are not limited as long as it is recognizable condition by observation of skin or a personal body feeling, but it is preferably by such an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.

An orally administered agent according to the present invention comprises ornithine or a salt thereof, and may include one or more pharmaceutically acceptable carriers, as necessary, and another active ingredient for other medical treatments as necessary.

An orally administered agent according to the present invention can be produced by mixing ornithine or a salt thereof with carriers and the like as necessary, according to an arbitrary method well known in the technological field of pharmaceuticals.

When an orally administered agent according to the present invention is formulated, additions such as an excipient, a binder, a disintegrating agent, a lubricant, a dispersant, a suspending agent, an emulsifier, a diluent, a buffering agent, an antioxidant, and an anti-bacterial agent can be added.

Dosage forms of the orally administered agent include tablets, powders, granules, emulsion, syrup, capsule and the like.

For instance, in the case of producing the orally administered agent in the form of tablets, powders, granules, or the like, the following can be added to make such formulation: excipients including a sugar such as lactose, white sugar, glucose, sucrose, mannitol or sorbitol; a starch such as potato, wheat or corn; an inorganic compound such as calcium carbonate, calcium sulfate, sodium bicarbonate or sodium chloride; and a plant powder such as licorice powder or gentian powder; disintegrating agents such as starch, agar, gelatin powder, cellulose crystal, sodium carmellose, calcium carmellose, calcium carbonate, sodium bicarbonate and sodium alginate; lubricants such as magnesium stearate, talc, hydrogenated plant oil, macrogol and silicon oil; binders such as polyvinyl alcohol, hydroxypropylcellulose, methylcellulose, ethylcellulose, carmellose, gelatin, and starch glue solution; surfactants such as fatty acid ester; plasticizers such as glycerin and the like,.

In case of producing the orally administered agent in the form of a liquid preparation such as syrup and the like, the following can also be added to make such formulation: water; sugars such as sucrose, sorbitol and fructose; glycols such as polyethylene glycol and propylene glycol; oils such as sesame oil, olive oil and soybean oil; preservatives such as p-hydroxybenzoic ester; flavors such as strawberry flavor and peppermint flavor and the like.

Although the concentration of ornithine or a salt thereof can be properly selected in accordance with a kind of the orally administered agent and an effect expected by administration of the orally administered agent, it is normally in a range of 0.1 to 90% by weight of ornithine or a salt thereof; preferably in a range of 0.5 to 80% by weight, and most preferably in a range of 1 to 70% by weight.

Although a dosage of the orally administered agent of the present invention may vary depending on such factors as an administration form, and age and body weight of the person who is being administered to, for an adult per day, it is normally in a range of 50 mg to 30 g of ornithine and a salt thereof, preferably in a range of 100 mg to 10 g, and most preferably in a range of 200 mg to 3 g, which is administered one time or separately a few times. Although a period of administration is not limited, it is normally in a range of one day to one year, preferably in a range of one week to three months.

A food additive according to the present invention can be prepared by the same method applied for the above orally administered agent. The food additive is normally mixed or resolved with another food additive as necessary and processed into, for example, powders, granules, a pellet, a tablet or various solutions.

As the food or drink according to the present invention, mention may be made of the food or drink comprising ornithine or a salt thereof, or a food additive according to the present invention.

The food or drink according to the present invention can be processed and manufactured by a general food and drink manufacturing method except that ornithine or a salt thereof, or the food additive according to the present invention is added to the known food or drink.

The food or drink according to the present invention can be manufactured by granulation methods such as a fluidized-bed granulation, a stirring granulation, an extrusion granulation, a rolling granulation, an air stream granulation, a compression molding granulation, a disruption granulation, a spray granulation or a blasting granulation; coating methods such as a pan coating, a fluidized-bed coating and a dry coating; an plumping method such as a puff drying method, an excess steam method, a foam mat method or a microwave heating method; or an extrusion method using, for example, an extruding granulator or an extruder.

The food or drink according to the present invention includes juices; soft drinks; teas, dairy products such as lactic acid bacteria beverages, fermented milk, frozen dessert, butter, cheese, yogurt, processed milk and defatted milk; animal meat products such as ham, sausage and hamburger; fish cake products such as plate-like fish cake or kamaboko in Japanese, pipe-like fish cake or chikuwa in Japanese, and fried fish cake or satsumaage in Japanese; egg products such as rolled egg with soup or dashimaki in Japanese and egg-tofu; confectioneries such as cookie, jelly, chewing gum, candy and snack; breads; noodles; pickles; smoked food products; dried fishes; fishes boiled in soy sauce or tsukudani in Japanese; salt curing products; soups; condiments; or any other forms.

Further the food or drink of the present invention may take the forms of powdery foods; sheet-like foods; bottled foods; canned foods; retort foods; capsule foods; tablet-like foods; fluid foods; nutritious supplement drinks or the like.

The food or drink according to the present invention can be used as health foods; functional foods; nutritious supplement foods; or food for specified health use, for improving skin condition.

A food additive such as a sweetener, a coloring agent, a preservative, a thickening stabilizer, an antioxidant, a color developing agent, a bleaching agent, a fungicide, a gum base, a bitter agent, an enzyme, a wax, a sour agent, a seasoning, an emulsifier, a nutrient supplement, an additional materials for preparation, a flavor or a spice extract, which are generally used in a food and drink, can be added to the food or drink or the food additive according to the present invention.

Although the concentration of ornithine or a salt thereof to be added can be properly selected in accordance with a kind of a food or drink, and an effect expected by administration of the food or drink, it is normally in a range of 0.1 to 90% by weight of ornithine or a salt thereof; preferably in a range of 0.5 to 80% by weight, and most preferably in a range of 1 to 70% by weight.

Although an intake of the food or drink according to the present invention may vary depending on an ingestion form, and age and body weight of the person being ingested to, for an adult per day, it is normally in a range of 50 mg to 30 g of ornithine or a salt thereof, preferably in a range of 100 mg to 10 g, and most preferably in a range of 200 mg to 3 g, which is ingested one time or separately a few times. Although a period of ingestion is not specified, it is normally in a range of one day to one year, preferably in a range of one week to three months.

The followings are test examples of the effect of orally ingested ornithine on improving skin condition.

### TEST EXAMPLE:

Six tablets of example 1 (the tablet containing ornithine) or 6 tablets of comparative example 1 (the tablet not containing ornithine) were ingested to two groups with 7 subjects out of 14 normal subjects of each male and female between 45 to 64 years of age a day for 3 weeks.

Before the ingestion and just after the ingestion, the improvements in skin condition of each subject were evaluated using Visual Analogue Scale (VAS) method.

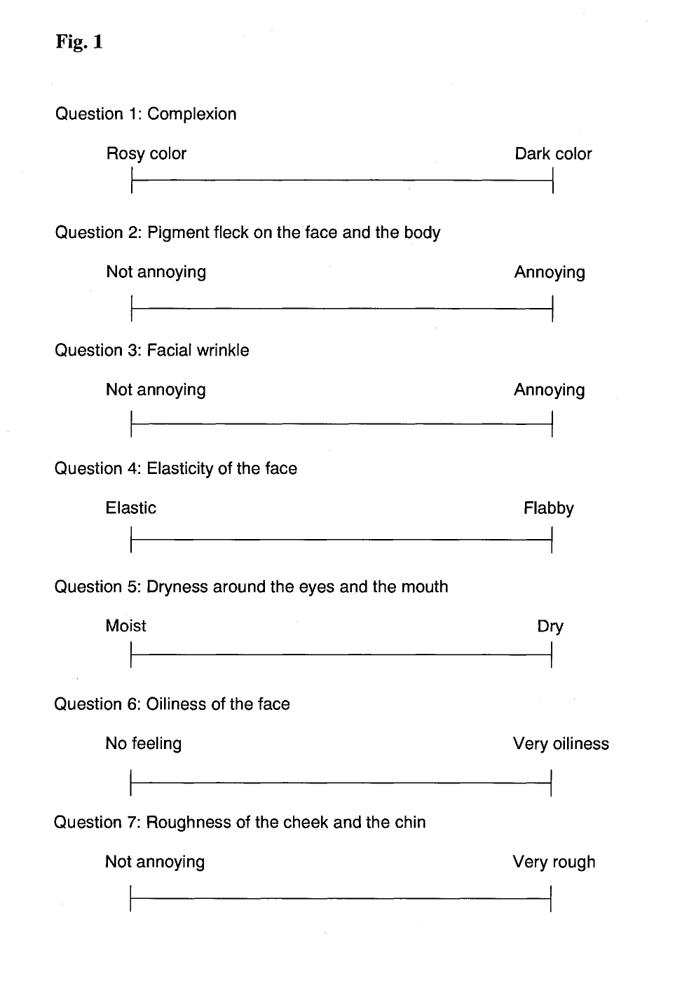

Specifically each end of the segment has a criterion of expression. Referring to FIG. 1, each subject marked somewhere in the line, corresponding to each term of the questionnaires. The distance (mm) from the left end of the line to the marked point was measured and the difference between before and after the test was calculated. The difference by the value before the test was shown in the percentage calculated; and each average value and standard deviation for each group were calculated. Further the average improvement ratio (%) was the value obtained by subtracting the average value of the placebo group from the average value of ornithine group. Further, it was confirmed that there was no difference between two groups before the administration test.

Further the test was carried out under a random assignment and the comparison between the double blind parallel groups was carried out. The test of the statistically significant difference between two groups was an unpaired t-test of both side distributions using the difference between the beginning and just after the test.

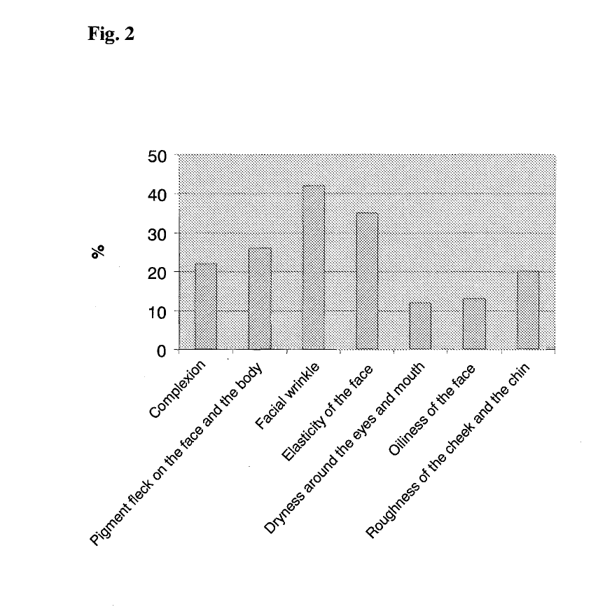

The results are shown in FIG. 2. The effects of orally ingested ornithine are shown in all categories and especially there are significant differences between a group of ornithine and a group of placebo in two categories of complexion and facial wrinkle.

From the above results, it is obvious that orally ingested ornithine can improve skin conditions.

The followings are the example of the present invention.

### Example 1

### Production of a Tablet Containing Ornithine

A mixture of 136.2 Kg of ornithine hydrochloride (Commercial name: L-ornithine hydrochloride, Kyowa Hakko Kogyo Co., Ltd.); 36.0 Kg of a fine cellulose crystal (Commercial name: Avicel FD101, Asahi Kasei Chemicals Co., Ltd.); 6.6 Kg of sucrose fatty acid ester (Commercial name: DK ester F-20W, Daiichi Kogyo Seiyaku Co. Ltd.); 1.2 Kg of calcium phosphate (Commercial name: Tricalcium phosphate, Taihei Chemical Industrial Co., Ltd.); and 20.0 Kg of β-cyclodextrin (Commercial name: Seldex B-100, Nihon Shokuhin Kako Co., Ltd.); was mixed using a conical blender (CB-1200 Blender, Nihon Kansoki Co., Ltd.). The mixture obtained was compressed and molded to a tablet of 250 mg with 8 mm of diameter under 10 KN of compression-molding pressure using the rotary compression molding machine (VIRG0524SS1AY, Kikusui Seisakusyo Ltd.).

### Example 2

### Production of an Enteric Capsule Containing Ornithine

A mixture of 20 Kg of the mixture prepared in Example 1 and 0.2 Kg of silicon dioxide was mixed and stirred. The mixture obtained was put into a capsule-filling machine to fill 20,000 tablets of gelatin Number 2 hard-capsules to provide the hard-capsules. The surfaces of the hard-capsules obtained were coated with a zein solution using High Coater HCT-48 (Freund Corporation) to produce 20,000 enteric capsules containing ornithine hydrochloride.

### Example 3

### Production of Enteric Tablet Containing Ornithine

The surfaces of the tablets prepared in Example 1 were coated with shellac solution using High Coater HCT-48 (Freund Corporation) to produce an enteric tablet.

### Example 4

### Production of a Drink Containing Ornithine

Each 1.28 Kg of ornithine hydrochloride (Commercial name: L-ornithine hydrochloride, Kyowa Hakko Kogyo Co. Ltd.); 3 Kg of erythritol (Nikken Kagaku Co. Ltd.); 0.05 Kg of citric acid (Kyowa Hi Foods Co. Ltd.); 3 g of artificial sweetener; and 0.06 g of flavor were stirred and dissolved in 50 L of water at solution temperature 70° C.; After the pH of the solution was adjusted to 3.3, the solution was sterilized using plate sterilization and filled into bottles. The bottle was sterilized using a pasteurizer to produce the ornithine beverage.

### Comparative Example 1

Instead of ornithine hydrochloride in Example 1, the same amount of lactose was used to produce a tablet not containing ornithine.

While the invention has been described in detail and with reference to specific embodiments thereof, it will be apparent to one skill in the art that various changes and modifications can be made therein without departing from the spirit and scope thereof. All references cited herein are incorporation in their entirety.

What is claimed is:

1. An orally administered agent for improving skin condition which comprises ornithine or a salt thereof as an active ingredient.

2. The agent according to claim 1, wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.

3. A food and drink, or a food additive for improving skin condition which comprises ornithine or a salt thereof as an active ingredient.

4. The food and drink, or the food additive according to claim 3, wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.

5. A method for improving skin condition which comprises administering or ingesting ornithine or a salt thereof to a subject in need thereof.

6. The method according to claim 5, wherein the improvement in skin condition is at least one improvement in skin condition selected from the group consisting of an improvement of complexion, an improvement of pigment fleck on the face or the body, an improvement of wrinkle in the face, an improvement of elasticity of the face, an improvement of dryness of the skin, an improvement of oiliness of the face and an improvement of roughness of the cheek and the chin.
